(19) United States
(12) Patent Application Publication
(10) Pub. No.: US 2015/0265797 A1
(43) Pub. Date: Sep. 24, 2015

(54) Field-effect Harmonizer for biosystems and substances

(71) Applicant: Gennady Vasilievich Vorsunov, Serpukhov Moskovskaya Obl. (RU)

(72) Inventor: Gennady Vasilievich Vorsunov, Serpukhov Moskovskaya Obl. (RU)

(21) Appl. No.: 14/434,096

(22) PCT Filed: Apr. 29, 2011
(86) PCT No.: PCT/RU2011/000292

§ 371 Date: Apr. 7, 2015

(30) Foreign Application Priority Data
Apr. 25, 2011 (RU) 2011115987

(51) Int. Cl. A61M 21/02; A61N 5/06

(52) U.S. Cl. CPC A61M 21/02; A61N 5/0618; A61N 2005/063; A61N 2005/067

(57) ABSTRACT

A field-effect harmonizer for biosystems and substances with a wide application range in a plurality of shapes and sizes from packaging for perfumery products, water, foodstuffs, alcoholic products, and medications to chambers and facilities for healthcare and biotechnology problem solving. The invention generates fields with a positive impact on living organisms including human bodies and on substances, comprising a disc with 23.5 or more GB of G. Vorsunov's biologically active films (BAF) or a disc with film plates affixed thereof, obtained from the discs that had been recorded with BAF, or from several such plates compiled in stacks, or from other data carriers storing information in a quality fashion and in an amount sufficient for launching positive changes in biosystems, located on any surface covered with a light-reflecting material made of aluminum foil, mirror, or polished metal, etc., to reflect the field to the biosystem.

### CROSS-REFERENCE TO RELATED APPLICATION

This present application claims the benefit of PCT Application Number PCT/RU2011/000292 filed Apr. 29, 2011 and published on Nov. 1, 2012 as WO2012/148302. The earliest priority filing date claimed is Apr. 25, 2011.

### FEDERALLY SPONSORED RESEARCH

Not Applicable

### SEQUENCE LISTING OR PROGRAM

Not Applicable

### BACKGROUND

The present invention relates to the field of biotechnology, healthcare and ecology technologies and is intended for generating a biologically active field with relatively effective power sufficient for changing the state of the human or animal body, or other biosystems, from a disharmonious diseased state into a harmonious healthy state; promoting emotional well-being and raising energy levels; slowing the aging process; helping the elderly to stay active; solving ecology problems; and improving consumer appeal of various substances and articles used by people throughout their lives. It may be applied to a broad range of biotechnological problems.

### Prior Art

All devices described below are sources of biologically actives fields, which harmonize the condition of biosystems, water, and other substances existing in liquid and semi-liquid states. The foundation for their creation are biologically actives films (BAF) by Gennady Vorsunov.

When transmitted to any screen (e.g., TV, computer), G. Vorsunov's biologically active films (BAF) generate fields in space that affect the biological fields of living organisms and change their state from a disharmonious (corresponding to an ailing organism) into a healthy harmonious state. Harmonization of the ailing person's bio-field triggers various biophysical, biochemical and other body processes, which resolve many health problems including some that are difficult or impossible to resolve with traditional medicine. Copyrights to these films and to their use, as well as the description are registered as follows: G. Vorsunov, Copyright, THREAD ID 1-70 HVHU, THREAD ID 1-70JF6Y, Jun. 15, 2010.

One of the ideas used for making these films is patented by G. V. Vorsunov in Germany, useful model patent # 21 2008 000 034.2., 03.04 2010. The device described in this known patent is a prototype for the development of the devices of the present invention. The disadvantage of this prototype is its large size, which limits its application.

All the devices were tested on volunteers with chronic or acute medical problems. In most cases, positive results were obtained in the following areas: chronic joint pain, heart and head ache; rehabilitation following traumatic face and head injury; rehabilitation of infants and children following birth trauma: a girl suffering from cerebral palsy showed improved speech and motor coordination; improved logical skills and better memory in the elderly; eliminating meteo sensitivity, etc.

Furthermore, in March 2011, at the request of G. Vorsunov, the scientists at the OOO KTI laboratory (Saint Petersburg, Russia, www.kti.spb.ru, e-mail: info@kti.spb.ru), using the GDV camera experimentally demonstrated that water responds to the fields emitted by the harmonizers of G. Vorsunov's biosystems. Also, with Korotkov's GDV camera, G. V. Vorsunov registered reactions of people, toothpaste, and creams to these fields. Reactions of sick patients' bodies to the fields generated by BAF and the proposed devices are demonstrated by filming the trouble spots in the IR region during the fields' application to the organisms. An IR camera with 0.05 degr. C. sensitivity recorded a decrease in body temperature at trouble spots by 0.5 and more degrees 15-30 min. post treatment with the proposed device and BAF.

The fields from the proposed devices are recorded on an instrument for measuring geophysical anomalies (IGA1) by Yu. P. Kravchenko (www.iga1.ru). In his article “Experimental Measurement of the Quantum Mechanics Wave Function,” on p. 22, G. I Shilov described his experiments measuring emission of torsion fields on the present device (article at http://.spinor. kiev.ua). P. Garjaev, Doctor of Biology, (http://p-gariaev.narod.ru/wave_gen_intro.htm) views the human genome as a quantum biocomputer performing spin-torsion recording and storing of information and its transfer to cells. He created an active model of a biocomputer in which information is read and transferred by laser beam. Information obtained in this model is used as a means for programming body cells for rejuvenation, healing, etc. G. Vorsunov used laser's ability to capture and transfer information in the devices NQ8 and N9.

Changes in the field areas recorded by the GDV camera for water, toothpaste, creams, and humans and a decrease in body temperature at the anomalous zones of the body under the effect of the fields from the devices of the present invention are a reliable proof of the existence of these fields and of their performance, while the improvement in health of the device users and BAF users confirm their positive impact on human bodies (and also on dogs and cats).

### DISCLOSURE OF THE INVENTION

The technical result of the present invention is to design technologies for healthcare and ecology, the biotechnologies combining high performance with low cost, simplicity and easy operability and absolute safety.

Said technical result is achieved by a field-effect disc harmonizer for biosystems and substances, to generate fields with a positive impact on living organisms including human bodies and on substances, comprising a disc with 23.5 or more GB of G. Vorsunov's biologically active films (BAF) or a disc with film plates affixed thereof, obtained from the discs that had been recorded with BAF, or from several such plates compiled in stacks, or from other data carriers storing information in a quality fashion and in an amount sufficient for launching positive changes in biosystems, located on any surface covered with a light-reflecting material made of aluminum foil, mirror, or polished metal, etc., to reflect the field to the biosystem. Furthermore, the field-effect harmonizer for biosystems and substances comprises cell structures that can be made in a plurality of shapes but with the following common features: the exterior cell contour has to be a regularly shaped polygonal prism or a cylinder wherein cylinders or prisms of smaller sizes, both intersecting and not intersecting and having light-reflecting coatings or polished surfaces are symmetrically positioned inside the cells relative to their axis, wherein the devices according to paragraph 1 positioned on the outer side of the cells relative to the biosystem and lenses, cones, or pyramids made of glass, crystal, or minerals located inside the cells and with their pointed end directed toward the biosystem, and having a field reflector common to the entire device or separate for every cell, made of aluminum foil or of mirrors reflecting the field to the biosystem.

One or more fiber optical bundles are fixed on the surface of the device with one or both ends, and subsequently, the harmonizers according to paragraph 1 or 2 or a combination thereof along with the fixed ends of the optical fiber are either fixed on the surface with a light-reflecting coating, wrapped in aluminum foil, or placed into a box with mirrored interior walls or with another reflective surface, while the optical fiber outside the box or foil is either loose, and in that case it is used to transport the field to one biosystem or one substance, or it is split into several bundles and in that case the field is transported to several biosystems or several substances simultaneously; or it is spread and fixed on the mirrored surface or any other emission surface covered with a light-reflecting coating; or it is evenly wrapped around a frame followed by a fixation on the optical fiber layer of the reflector made of mirrors or aluminum foil and reflecting the field into the inner space defined by the fiber, containing the biosystem in need of the field exposure.

The devices according to paragraph 1, 2, or 3 in various embodiments corresponding to the problems to be solved and technical possibilities of their fixation and exploitation are fixed on the surface of any revolving parts or inner spaces thereof, such as blades, rotors of air fans, air and water generators, water propellers of ships, wheels of vehicles, automobile and other vehicle tires, revolving elements of the devices specifically manufactured to generate and emit into the space biologically active fields with a positive effect on biosystems and substances.

### SUMMARY

A field-effect harmonizer for biosystems and substances with a wide application range in a plurality of shapes and sizes from packaging for perfumery products, water, foodstuffs, alcoholic products, and medications to chambers and facilities for healthcare and biotechnology problem solving, is any space defined by surfaces covered with light-reflecting materials housing the devices according to paragraph 1, 2, 3, or 4 in combinations corresponding to the problems being solved with the device of the present invention.

In the field-effect harmonizer, the ends of the optical fiber bundles are fixed in the device according to paragraph 5.

A video image of BAF or a laser beam, or several beams, or BAF modulated with a video image, or unmodulated with BAF from the laser beam (laser beams) located inside the device according to paragraph 5 or 3 is transmitted through a projector lens onto the optical fiber inlet with a side-emitting effect.

A laser beam or laser beams from the field-effect harmonizer for the biosystems and substances located inside a box is sent through an opening in the chamber wall to the biosystem in need of exposure.

### DETAILED DESCRIPTION

### Embodiment

1. A field-effect harmonizer for biosystems and substances is a disc with 23.5 or more GB of G. V. Vorsunov's BAF with a reflector made of aluminum foil or flat or parabolic mirror pasted onto its inactive side (on the side of the label,) and film plates obtained from the discs that had been recorded with BAF prior to removing the plates, fixed onto the work surface of the disc.

As G. Vorsunov's experiments demonstrate, the effect of these devices is directly proportional to the amount of information stored in the device and efficiency of BAF themselves.

To achieve results in solving health problems, it is sufficient to place the device of the present invention with the reflector facing out, onto a patient's body in the anomalous zone. In a preferred embodiment, the recorded plates are pasted onto a base made of thin flexible plastic that had been previously covered with aluminum foil. Complicated cases require stacks of many plates. These devices can be manufactured in any size by affixing stacks of 5-10 or more plates (240 GB of BAF information) next to each other onto a light-reflecting surface of a required size. In several days or several months of continuous daily use of these devices, health problems disappear completely or partially. (At night, the device is placed under the pillow cover, under sheets, etc., and during the day, the device is placed on the body in trouble spots. They are especially effective in children. For relatively uncomplicated problems, such as pain from acute gastritis, monthly cramps in women, discomfort following alcohol intake, spontaneous headaches, pain in the kidneys, liver, heart etc., it is sufficient to keep the device in the corresponding area for 30-60 min., and the pain and discomfort diminish. Chronic problems require treatments over several months. The device of the present invention solves health problems in animals.

Availability of Blue ray discs with 320 GB recording capacity and holographic discs for 1.6 or more GB (such discs were developed in Japan), the process of making powerful devices of this type will become much less complicated, and the devices themselves will become more compact.

Emission from disc and film plate surfaces obtained from the discs after they had been recorded with BAF can be attributed to the fact that during recording of G. Vorsunov's biologically active films (BAF) on discs on the nanometer level, the film, that is an information carrier is burned out by a laser with a harmonious three-dimensional structure shaped as a helix, which reflects information from BAF. The torsion field emitted from this structure carries the BAF information producing various biophysical, biochemical and other processes in the body and promoting good health. Due to the small helix pitch distance, when recording is done in the Blue-Ray format, the helix approaches the shape of a concentric circle system, and according to the data from G. I. Shilov, a Russian scientist, (www.shipov.com), (www.fizvakum.narod.ru), chapter 4, and A. R. Pavlenko, a Ukrainian scientist, p. 180, A. R. Pavlenko “Computer, Mobile Phone, and . . . Health? 2007, Ukraine, Kiev, Osnova ISBN 978-699 -266-9, the circle has the highest torsion contrast ratio (http://www.spinor.kiev.ua). Field emission from the film surfaces following recording of BAF thereupon also explains “The Cavitary Structure Effect” coined by the Russian entomologist V. S. Grebennikov (http://www.incognita.rU/hronik/planet/p 020.htm). The cavitary structure effect known to all is demonstrated by the fields emitted by honeycomb frames from beehives, emptied of honey and stacked one on top of the other.

According to the theory of torsion fields, they don't carry energy but information. To protect people from the adverse torsion fields emitted by computers, phones, etc, A. R. Pavlenko developed the device called Forpost 1, United States patent Pavlenko, et al U.S. Pat. No. 6,548,752 Apr. 15 2003.(www.spinor.kiev.ua)

The mechanism by which information affects living organisms was explained by professor Masuru Emoto of Japan (www.masaru-emoto.net, www.hado.net). Based on numerous experiments, he had proved that under the influence of information, the structure of water changes proportionately to the type of the information received. Since the human body, as well as all biosystems, mostly consists of water, any positive external information affecting a living organism transforms it from a diseased to a healthy state. Experiments studying reaction of water to various external factors were conducted by K. G. Korotkov, Doctor of Engineering (www.korotkov.org). In his experiments, he demonstrated that human consciousness (I.e. the field that a person consciously generates and emits) affects the state of water. The discs of the device according to paragraph 1 contain recordings of biologically active films, i.e. information aimed at maintaining a harmonious and healthy state of living organisms. Accordingly, impacted by this information, the water in living organisms changes its structure proportionally to the received information, thus transforming the organisms from a diseased to a healthy state

The advantages of the device of the present invention are the simplicity of manufacture and use thereof; it may be made with a variety of modifications both in size and the relative effective power by varying the amount of information stored in the device. For example, for leg and arm joints, it is sufficient to use one stack of films with BAF information or one disc, while to restore the brain function, it is effective to use at least three discs or BAF information plate stacks. This affords consistently high

efficacy in situations with different degrees of complexity. In order to cover a wide variety of areas, the device according to paragraph 1 must utilize a large number of discs and plates obtained from discs after they had been recorded with information from BAF, and the single primary source of the biologically active field in this device is BAF information. This limits its possibilities.

2. In order to eliminate this disadvantage, we designed a field-effect cellular harmonizer for biosystems as various combinations of symmetrical regularly shaped cells in the form of cylinders, of prisms, wherein cylinders located inside the cells are both intersecting and not intersecting and have light-reflecting coatings and of discs or stacks of plates with BAF recordings according to paragraph 1 and also of lenses, cones, or pyramids made of glass, crystal, or natural or manmade minerals; having a field reflector common to the entire device or separate for every cell reflecting the field into the work zone of mirrors or foil.

As G. Vorsunov'e experiments demonstrated, the devices composed of cells in the shape of symmetrical hexagonal prisms or cylinders comprising cylinders of lesser diameters in different configurations but symmetrically arranged and covered with light-reflecting coatings emit a biologically active field of relative effective power sufficient for making a positive impact on the condition of the human body. (These devices can be made in different sizes, from nanometers to meters and bigger). The cells can be made in an infinite plurality of shapes and there can be a multitude of different embodiments of such devices, from jewelry, clothes, rugs, hats made of thread or cord with light-reflecting coatings (such as aluminum foil) to various panels, structural elements of furniture—beds, armchairs, various cars and buildings, in combination with BAF information carriers (discs, plate stacks according to paragraph 1, other information carriers) and with lenses, pyramids, cones made of glass or from various natural or manmade minerals, which fix the fields and improve the efficacy of the devices. It is particularly important to use these devices in the facilities for children and medical facilities. Emissions from these devices are generated due to the aforementioned “Cavitary Structure Effect” and also due to the shape effect described by G. I. Shipov, chapter 4 “On Shape Effect.”(www.fizvakum.narod.ru). The Shape Effect is also reported by another Russian scientist, A. Ye. Akimov (A. Ye. Akimov “The Physical Basis of the Fundamental Angi concepts”//Live Ethics, Science, Society: a compiled publication by A. Ye. Akimov, RANS academician, Director of the International Institute of Theoretical and Applied Physics./Penza 2000, pp 12-2.

The mechanism of the impact on living organisms and substances is the same as for the device according to paragraph 1. The device must be placed in the closest proximity to the user's body in such a way as to direct the field's emission toward the body. Relative effective power of the fields generated by such device is higher than that from device Ne1 due to the accumulative effect of the fields from all the sources. Efficacy of such devices is proportional to the number of cells and to the volume occupied by the entire structure, and also to the number of utilized devices according to paragraph 1 and the lenses, cones and pyramids made of glass or minerals that bring these fields into focus.

3. In order to eliminate the disadvantages inherent to the devices according to paragraph 1 and 2, we designed the “optical field-effect harmonizer for biosystems” comprising one or several optical fiber bundles (inflexible or flexible), wherein one or both ends are fixed on one or several devices according to paragraph 1 or 2, or on any combinations thereof, wherein said devices (or device) with the ends of the optical fibers affixed thereto are wrapped into foil or placed into a box coated with a light-reflecting material, or affixed to any light-reflecting surface, and optical fibers are either loose or also affixed to any base with a light-reflecting surface. The use of optical fibers facilitates the split of the bundles and can produce a simultaneous effect from the combination of devices according to paragraphs 1 and 2 or from one of the devices onto several biosystems. This is particularly important for big medical facilities. Said devices can be prepared and used in an infinity of ways: as rugs and bed covers, as parts of clothing, armchairs, beds, rooms, and as transportation devices from baby carriages to airplanes. In biotechnologies for transmitting BAF fields into required zones, etc., in his experiments, G. Vorsunov uses flexible optical fibers with 0.15 mm to 1 mm gauge and 0.1 m to 30 m length, and plate stacks of BAF recordings containing from 96 GB to 3 TB of information. In said devices, the light-reflecting surface reflects the fields from the films, and these fields penetrate the optical fiber. As demonstrated in practical applications, the fields transported from the device according to paragraph 1 through the fiber are emitted through the fiber's side surfaces. Optical fibers greatly increase the light-reflecting surface and accordingly, the relative effective power of the device in comparison to devices #1 and 2. This was confirmed in the experiments with water conducted in OOO KTI. Device Ns1 that had been tested at the OOO KTI facility contains 96 GB of information and consist of a disc and three films with BAF recordings fixed thereto. Devices Ns2 and #3 also contain a disc and three films and also carry 96 GB of information, but the plates of device #2 are fixed on a cellular structure, while the film in device Ns3 has 300 optical fibers with 0.15 mm gauge and 200-250 mm length fixed thereto. The reaction of water to the fields emitted by the device according to paragraph 3 is stronger than the reaction to the fields from the first device. Thus, the fields emitted from the surface of the plates storing BAF information were demonstrated to penetrate the optical fiber and while passing therethrough emitted into the space. The device users showed enhanced reactions of the organism as the number of fibers of the emitting fields increases.

An integrated effect on many organs and systems and on the whole body, if necessary, or on several people simultaneously can be achieved by using only one stack of films storing BAF information when placing the fibers next to the user's body by way of different constructions (flat or three-dimensional) with fibers fixed to the interior surfaces thereof. Varying the amount of information stored in the device according to paragraph 1, using the various devices according to paragraph 2, and varying the total area of the cross-section and the length of the optical fibers transmitting the field, the parameters of the field emitted by the device can be adjusted to achieve the highest efficiency. Using a large number of long fibers (placing them on the bottom of bodies of water or hanging them on floats at the desired depth) can effectively impact the state of the biosystems in river basins, lakes and seas.

Device Ne4. The disadvantage of the device according to paragraph 3 is the difficulty of managing an effective impact on biosystems in a large space. To eliminate this disadvantage we designed the “dynamic harmonizer for biosystems,” which can be any rotating object with the devices according to paragraphs 1, 2, and 3 fixed thereupon in any combinations that would actually be technically feasible. Common fans were used in the experiments. Their blades were wrapped into aluminum foil, and then plates with BAF recording were pasted onto the blades. Essentially, this is a rotating version of the device according to paragraph 1. As the experiments demonstrated, the rotating blades of such fans generate a field with biological activity much higher than the field emitted by the same blades when the fan is off. The higher the rotating speed, the higher the biological activity of the fields generated by such device. This phenomenon can be attributed to the torsion field emitted by any rotating object. Rotation of sources of biologically active fields generates additional torsion fields, which augment the filed emitted by said sources. That was demonstrated in the experiments conducted at OOO KTI. The reaction of water from a rotating fan with the device according to paragraph 1 affixed thereto is much stronger that that from a stationary device according to paragraph 1. Such fans achieve two goals: air circulation and a simultaneous generation of a biologically active field into a large space to solve health problems of the people in the facility where the fan is located. Such fans are especially useful in the ventilation systems of intensive care units, in hospices, in the facilities for children, old-age homes, and in the hospitals. They are also useful in people's homes, industrial facilities, and in all modes of transportation as prophylaxis against illnesses, for fostering emotional well-being and raising the energy level, etc. In addition, such devices are very useful in all technical devices where the devices according to paragraphs 1, 2, and 3 can be affixed to the moving parts or in the assemblies of said devices. First of all, this must be done inside automobile tires and on the windmill blades, on water generator's and screw propeller's axes. As the wheels, blades, or rotors with the information carriers containing BAF recordings affixed thereto rotate, biologically active fields are continuously emitted into the space, which positively affect the people inside these modes of transportation and all the living organisms in the vicinity of the operational automobiles or generators. In his experiment, G. Vorsunov pasted plates with BAF recordings on the inner surfaces of his automobile tires. Both people with health problems and the people inside the automobiles experience reactions similar to those from the devices according to paragraphs 1, 2, and 3. Considering that a huge amount of vehicles, wind and water generators, and other various mechanisms are always in motion, we can predict with certainty that these devices, when widely used, will be very effective for the preventive health care and for providing a good ecological environment along the roads and highways and near wind power plants. A natural movement of large amounts of air and water will broaden the area covered by these devices by many miles, and we can predict with certainty that an implementation of these devices into practice will make positive global effects on the ecology.

Device #5. A chamber with high density of biologically active fields for solving complex health problems (accelerating healing of vast wounds, in all kinds of acute insufficiencies, for helping children with a majority of problems including birth trauma and oncology, etc.), for the effect on various substances in order to improve their consumer appeal, such is perfumery products, water, fruit, vegetables, liquid and semiliquid food products and food products containing water as well as in biotechnologies for the acceleration of processes of cell division and cell augmentation, said chambers can be any closed space (box, room, building) with inner surfaces covered with light-reflecting coatings and comprising any of the aforementioned devices or a combination thereof. The impact on living organisms from such devices is much stronger and more effective than the impact from the devices according to paragraphs 1, 2, 3, and 4. This can be attributed to the fact that the fields emitted from the devices according to paragraphs 1, 2, 3, and 4 are contained within the space defined by the light-reflecting surface. Perfumery products kept in such devices for 3 or more days, exhibit a much more refreshing and rejuvenating effect than the initial perfumery products. Thus, it is very important to use this device for packaging of perfumery products. Water, fruit, and liquid products kept in a refrigerator made in the form of such device (the harmonizer according to paragraph 3 is placed in the refrigerator and its inner surfaces are covered with foil) have better taste. Cut up potatoes acquire a dark color faster. Theoretically, medications kept in such chambers have to exhibit an enhanced healing effect. Reactions of toothpaste and skincare creams kept in the refrigerator for more than three to five days were recorded with a GMV camera.

6. The ends of the fiber optic bundles are fixed inside the device according to paragraph 5. Owing to the utilization of a large number of field sources, such devices can expand the range of problems that can be solved with the maximum efficiency.

7. Light harmonizer for biosystems. This device comprises a bundle or several bundles with BAF transmitted through a projector lens onto the inlet thereof. The fiber bundles can be places in one plane or in various three-dimensional structures complete with a reflector reflecting the field into a work zone. When a BAF image passes through the fibers, it emits a biologically active field through the side surface thereof, similarly to the device according to paragraph 3. Fiber optic bundles from one projector can be split and used for several people or used to transmit BAF fields into several work zones. Devices containing optic fibers with good side-emitting capacity can be concurrently used as light sources. This is especially useful in the hospitals, hazardous industries, etc.

8. A laser harmonizer for biosystems is either the device according to paragraph 3, wherein a laser beam or several laser beams modulated with BAF video images are sent to the fiber optic inlet; or a box containing the device according to paragraphs 1 or 2, additionally comprising one or more lasers in such a way that the laser beam/s enters the optic fibers. In this device, a laser beam captures the information emitted by the fields of the device according to paragraphs 1 or 2 and carries it through the optic fibers, whereas similarly to the device according to paragraph 3, a biologically active field is emitted though the side surfaces thereof initiating various positive process in the living organisms. An electromagnetic field radiation is one of the laser's physical properties. In his experiments in wave genetics, P. P. Garyaev, Doctor of Biology, uses this property of laser beams to capture information and to emit it into the space. Optic fibers transport laser beams with no losses to very long distances, which can generate large biologically active fields with high effective power in very large spaces and due to the split, transmit them to a large number of biosystems over long distances.

A device modulating a laser beam has not yet been created because of the complexity of its design, but in the practical application of BAF, there was a case when the TV of one of the BAF users broke and he watched the BAF exclusively on a DVD player. He still experienced the positive effects. This means that the field emitted by the laser in the player showing the BAF made a positive impact on the human body. (The user was an elderly man with bad memory). This practical experience and P. P. Garyaev's experiments are the basis for the creation of this device.

9. In the open laser harmonizer for biosystems, the laser beam (or beams) positioned inside a chamber, the inner walls thereof are covered with a light-reflecting coating, are transmitted through an opening in the chamber wall not to an optic fiber but to a space beyond the chamber and into the work zone, i.e. to a biosystem in need of an impact. The field in the chamber is captured by the laser beam/s and emitted into a space while affecting the biosystems adjacent to the beam.

This version of the harmonizer is simple, convenient, and can make an impact at long distances and in large spaces. It is efficient in curing illnesses and can be used in biotechnology and ecological biotechnology.

### INDUSTRIAL APPLICABILITY

This technical solution is industrially applicable; since the description and the useful model indicate the purpose thereof, it can be manufactured and used to make a field-effect disc harmonizer for biosystems and substances for generating fields with a positive effect on living organisms, it can be produced and reproduced while the distinctive features of the device help achieve the desired technical result, i.e. they are essential.

The device as described in every of the claims can be manufactured by the means and methods described for the prototype available to everyone before the date of priority of the invention. Thus, the claimed technical solution meets the patentability criteria “Industrial Application.”

The efficacy of all the proposed devices is easily confirmed in practice and those skilled in the art who are interested in the device can easily test them.

What is claimed:

1. A field-effect disc harmonizer for biosystems and substances, which is used to generate fields with a positive impact on living organisms including humans and on substances, comprising a disc with 23.5 or more GB of G. Vorsunov's biologically active films (BAF) or a disc with film plates affixed thereof, obtained from the discs that had been recorded with BAF, or from a plurality of such plates compiled in stacks, or from other data carriers storing information in a quality fashion and in an amount sufficient for launching positive changes in biosystems, located on any surface covered with a light-reflecting material made of aluminum foil, mirror, or polished metal, etc., to reflect the field to the biosystem.

2. The field-effect harmonizer for biosystems and substances of claim 1, wherein said harmonizer comprises cell structures that can be made in a plurality of shapes but with the following common features: the exterior cell contour has to be a regularly shaped polygonal prism or a cylinder, wherein cylinders or prisms of smaller sizes, both intersecting and not intersecting and having light-reflecting coatings or polished surfaces, are symmetrically positioned inside the cells relative to their axis, wherein the devices of claim 1 positioned on the outer side of the cells relative to the biosystem and lenses, cones, or pyramids made of glass, crystal, or minerals located inside the cells and with their pointed end directed toward the biosystem, and having a field reflector common to the entire device or separate for every cell, made of aluminum foil or of mirrors reflecting the field to the biosystem.

3. The field-effect harmonizer for biosystems and substances of claims 1, 2, or a combination thereof, wherein one or more fiber optical bundles are fixed to the surface of the device (or devices) with one or both ends and, subsequently the harmonizers of claim 1, or 2, or a combination thereof along with the ends of the optical fiber affixed thereto are either fixed on the surface with a light-reflecting coating, or wrapped in aluminum foil, or placed into a box with mirrored interior walls or another reflective surface, while the optical fiber outside the box or foil is either loose, and in that case it is used to transport the field to one biosystem or one substance, or it is split into several bundles, and in that case the field is transported to several biosystems or several substances simultaneously; or it is spread and fixed on the mirrored surface or any other emission surface covered with light-reflecting coatings; or it is evenly wrapped around a frame followed by a fixation on the optical fiber layer of the reflector made of mirrors or aluminum foil and reflecting the field into the inner space defined by the fiber, containing the biosystem in need of the field exposure.

4. The field-effect harmonizer for biosystems and substances of claim 1, 2, or 3, wherein the devices of claim 1, 2, or 3 in different combinations corresponding to the problems to be solved and technical possibilities of their fixation and exploitation are fixed on the surface of any revolving parts or inner spaces thereof, such as blades, rotors of air fans, air and water generators, water propellers of ships, wheels of vehicles, automobile and other vehicle tires, revolving elements of devices specifically manufactured to generate and emit into the space biologically active fields with a positive effect on biosystems and substances.

5. The field-effect harmonizer for biosystems and substances of claim 1, 2, 3, or 4 with a wide application range in the plurality of shapes and sizes, from packaging for perfumery products, water, foodstuffs, alcoholic products, and medications to chambers and facilities for healthcare and biotechnology problem solving, wherein said harmonizer can be any space defined by the surfaces covered with light-reflecting materials housing the devices of claim 1, or 2, or 3, or 4 in combinations corresponding to the problems being solved with the device of the present invention.

6. The field-effect harmonizer for biosystems and substances of claim 3, wherein the ends of the optic fiber bundles are fixed in the device of claim 5.

7. The field-effect light harmonizer for biosystems and substances of claim 3 combining the functions of the source of a biologically active field and a lighting system, wherein a video image of BAF is transmitted through a projector lens onto the fiber optic inlet.

8. The field-effect laser harmonizer for biosystems and substances of claim 3, wherein a laser beam or several laser beams modulated or unmodulated with BAF video images are sent to the fiber optic inlet from the laser/s located inside the device of claim 5 or 3.

9. The field-effect open laser harmonizer for biosystems and substances of claim 8, wherein the laser beam/s placed in a box is sent through an opening in the chamber wall to the biosystem in need of exposure.
